(19) United States
(12) Patent Application Publication
(10) Pub. No.: US 2010/0010317 A1
(43) Pub. Date: Jan. 14, 2010

(54) SELF-CONTAINED DATA COLLECTION SYSTEM FOR EMOTIONAL RESPONSE TESTING

(76) Inventor: Jakob DE LEMOS, Copenhagen V (DK)

Correspondence Address: PILLSBURY WINTHROP SHAW PITTMAN, LLP, P.O. BOX 10500, MCLEAN, VA, 22102, US

(21) Appl. No.: 12/170,041

(22) Filed: Jul. 9, 2008

(51) Int. Cl. A61B 5/00

(52) U.S. Cl. 600/300

(57) ABSTRACT

A system and method is provided for providing self-contained data collection systems for emotional response testing of one or more subjects based, for example, on eye properties of the subjects. More particularly, market research may be conducted through testing emotional responses of one or more subjects at one or more self-contained data collection systems. The emotional responses of the subjects may be tested based on eye properties that may be indicative of subconscious physiological reactions that evidence a given emotional response (e.g., blink rate, eye movement, pupil dilation, etc.). Administering tests at the self-contained data collection systems may include presenting one or more tests stimuli to the subjects (e.g., visual stimuli, auditory stimuli, etc.), wherein the self-contained data collection systems may have various systems operable therein to analyze properties of the subjects' eyes to determine whether and/or how the subjects emotionally respond to the stimuli and/or to transmit the data to another location for analysis.

### FIELD OF THE INVENTION

The invention relates to systems and methods for providing self-contained data collection systems for emotional response testing of one or more subjects based, for example, on eye properties of the subjects.

### BACKGROUND OF THE INVENTION

Market researchers often conduct surveys, questionnaires, and other tests to determine consumer preferences. In particular, market researchers typically conduct market research on behalf of “Consumer Packages Goods” companies (“CPGs”), among other entities. Often, market research testing will be done at a market research facility, where the market research companies recruit test subjects, sometimes meeting certain demographic or other requirements, and then pay the subjects for the time they take to participate in the test. Regardless of whether an entity hires a third-party market research firm or conducts its own market research, a significant expenditure of resources is often involved in setting up a test facility, recruiting test subjects that meet specific demographic profiles, and paying personnel to administer the tests. Additionally, because potential test subjects may be unwilling to travel long distances to a market research facility, it can often be difficult and/or costly to obtain test data from a large sample size of subjects. Often, to get an extensive set of data from a large number of test subjects, a market research company may need to build and operate many different test facilities in different geographic locations, and/or spend significant amounts to recruit test subjects.

The aforementioned drawbacks are not unique to commercial market research, moreover. In particular, similar drawbacks may apply whenever there is a need to identify and test subjects, regardless of the purpose of the test or testing method. For these and other reasons, various drawbacks exist with traditional market research models.

Emotional response testing for various purposes has generally become known. For example, emotional response testing may sometimes be used to conduct market research of consumers by or on behalf of providers of goods and/or services (e.g., CPGs). Additionally, emotional response testing can be used for various other purposes. For example, emotional response testing can also be used alone or in combination with “rational” response testing, which may be conducted using surveys, questionnaires, or other such methods. One technique that has recently become more feasible as a method of conducting emotional response testing includes measuring one or more eye properties of a subject (e.g., eye movement, blink rate, pupil dilation, etc.). One exemplary technique for conducting emotional response testing based on eye properties is disclosed in U.S. Patent Application Pub. No. 2007/0066916, entitled “System and Method for Determining Human Emotion by Analyzing Eye Properties,” the disclosure of which is hereby incorporated by reference in its entirety.

Various problems and drawbacks exist with known techniques for conducting market research and emotional response testing.

### SUMMARY OF THE INVENTION

The invention addressing these and other drawbacks of existing and known techniques for conducting market research includes testing emotional responses of one or more subjects at one or more self-contained data collection systems. In particular, the emotional responses of the subjects may be tested based on eye properties that may be indicative of subconscious physiological reactions (e.g., blink rate, eye movement, pupil dilation, etc.) as opposed to cognitive rational responses. Tests administered at the self-contained data collection systems may include one or more stimuli presented to the subjects (e.g., visual stimuli, auditory stimuli, olfactory stimuli and/or other stimuli). The self-contained data collection systems may have various systems operable therein to collect data from and/or analyze properties of the subjects' eyes to determine whether and/or how the subjects emotionally respond to the stimuli.

In general, any given entity desirous of determining an emotional response of one or more subjects may make use of the self-contained data collection systems, which may be distributed in any number of locations, to acquire emotional response data from any appropriate group of subjects (e.g., meeting certain criteria, across any number of demographics, and/or meeting some other criteria). For example, a CPG (consumer package goods) company, a market research company, or another entity may use the self-contained data collection systems to gather emotional response data from the subjects in relation to existing or proposed advertisements, to a new product or a new feature of a product, or to packaging for a product, among other things.

As used herein, a “subject” may refer, for example, to a respondent or test subject, depending on how the invention is used and from whom emotional response data and/or other data is to be collected. In any particular data collection session, a subject may comprise an active participant (e.g., responding to instructions, viewing and/or responding to various stimuli, whether visual or otherwise, etc.), or a passive individual (e.g., unaware that data is being collected). Other nomenclature for a “subject” may also be used depending on the particular application of the invention.

In one implementation, depending on the scope, subject matter, purpose, or other characteristic of a desired test, one or more subjects or groups of subjects may be targeted, designated, or otherwise selected based on one or more characteristics. For example, the subjects or groups of subjects may be sampled according to demographic characteristics (e.g., age, sex, ethnicity, nationality, sexual orientation, marital status, education level, income level, specified interests or preferences, city, state, or country of residence, etc.), physical characteristics (e.g., smell, hormone, pheromone, etc.), emotional profile (e.g., phobia, general emotional state, etc.), personality (e.g., a Myers Briggs personality type or cognitive style), disabilities (e.g., blind, deaf, etc.), professional characteristics (e.g., a type of license or affiliation), or any other suitable characteristic or combination of characteristics for which a test may be desired. Either or both of observed or declared behavior may be used.

As used herein, a “test” or “emotional response test” may generally refer to a wide variety of activities in which a subject may engage, either actively or passively (e.g., advertising or marketing studies). A “test” or “emotional response test” may include, at least in part, presenting the subject with any individual, series, or combination of test stimuli that may be presented to a subject for purposes of determining the subject's emotional response to the test stimuli.

As used herein, the “stimulus” or “stimuli” presented to the subject may comprise any fixed or dynamic stimulus or combination of stimuli relating to one or more of the subject's five senses (i.e., sight, sound, smell, state and/or touch). The stimulus may compromise any real stimulus, or any analog or electronic stimulus that can be presented to the subject via known or future-developed technology. For example, visual stimuli may include, but are not limited to, pictures, artwork, charts, graphs, text, movies, multimedia presentations, interactive content (e.g., video games), scents and/or other visual stimuli. The stimuli may be recorded on any suitable storage media may include live scenarios and/or real-time generation of the stimuli (e.g. scent).

As described in greater detail herein, the self-contained data collection systems may comprise any environmentally controlled data collection system, including kiosks, partially-enclosed areas (e.g., booths), fully-enclosed areas (e.g., enclosed structures, rooms, areas in a cinema, etc), or another structure or environment having one or more of the components described herein for emotionally testing subjects. In various implementations, the self-contained data collection systems may be arranged as stationary data collection systems, mobile data collection systems, or various combinations thereof. In one implementation, the self-contained data collection systems may be distributed at any number of various locations in any number of geographic regions (e.g., locally, nationally, internationally). For example, the locations may include, but are not limited to, retail stores, shopping malls or centers, airports, bus or train terminals, schools, government buildings, businesses, car dealerships, medical/clinical facilities, workplaces, homes, public parks or spaces, private spaces, or any other locations, without limitation, depending on the scope and subject matter of the desired testing. In one implementation, each of the one or more self-contained data collection systems may be designated to administer one or more tests for one or more targeted subjects or groups of subjects for one or more entities that desire information relating to a group of subjects' emotional responses to various stimuli. In one implementation, the self-contained data collection systems may be used to administer tests to a plurality of subjects in a substantially simultaneous manner.

Various other aspects, features, and advantages of the invention will be apparent through the detailed description of the invention and the drawings attached hereto. It will also be understood that both the foregoing general description and the following detailed description are to be regarded as exemplary only, and not restrictive of the scope of the invention.

### BRIEF DESCRIPTION OF THE DRAWINGS

FIG. 1 illustrates an exemplary system comprising a plurality of self-contained data collection systems for emotional response testing, according to one aspect of the invention.

FIG. 2 illustrates an exemplary self-contained data collection system for emotional response testing, according to one aspect of the invention.

FIG. 3 illustrates various exemplary application modules that can enable the various features and functions of the invention, according to one aspect of the invention.

FIG. 4 illustrates an exemplary method for operating a plurality of self-contained data collection systems for emotional response testing, according to one aspect of the invention.

### DETAILED DESCRIPTION OF THE INVENTION

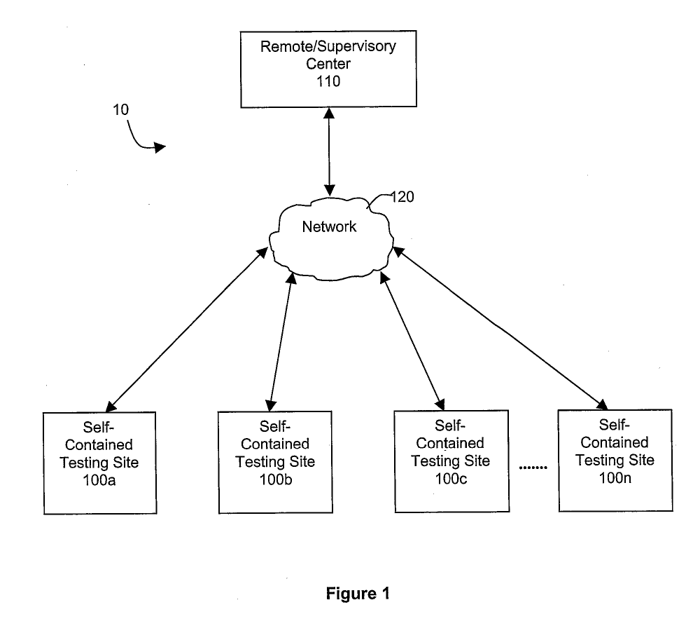

FIG. 1 illustrates an exemplary system 10 comprising a remote supervisory center 110 in operative communication with a plurality of self-contained data collection systems 100a, 100b, 100c, . . . 100n. The self-contained data collection systems 100a-n may communicate with the remote supervisory center 110 over a wired or wireless network 120 using any suitable communication link. According to one aspect of the invention, various tests, including emotional response tests, may generally be administered to one or more subjects at the plurality of self-contained data collection testing systems 100a-n. In one implementation, the self-contained data collection testing systems 100a-n may be designed to administer emotional response testing of the subjects based on measurements of eye data (e.g. via an eye tracking device). For example, the self-contained data collection testing systems 100a-n may have various systems operable therein to analyze parameters relating to the subjects' eyes to determine whether and/or how the subjects emotionally respond to one or more stimuli presented to the subjects during the tests administered at the self-contained data collection testing systems 100a-n.

According to one aspect of the invention, the self-contained data collection systems 100a-n and the remote supervisory center 110 may cooperate in various customizable configurations to collect data and/or determine the emotional responses of one or more subjects to various stimuli presented to the subjects during tests administered at the self-contained data collection systems 100a-n. For example, in various implementations, and as will be discussed in greater detail below, some, all, or any combination of the functions described herein (e.g., subject authentication, data collection, data analysis, report generation, etc.) may be performed at either or both of a self-contained data collection system 100 or the remote supervisory center 110 and/or elsewhere. Similarly, some, all, or any combination of the data or information described herein (e.g., information on tests, stimuli packages, test subjects, collected emotional response data, reports, etc.) may be stored at a self-contained data collection system 100, the remote supervisory center 110, and/or other locations. As such, any particular configuration for the 10 system described herein may be employed.

In one implementation, remote supervisory center 110 may comprise, include, or interface with at least one server. The server may include, for instance, a workstation running Microsoft Windows™ NT™, Microsoft Windows™ 2000, Unix, Linux, Xenix, IBM, AIX™, Hewlett-Packard UX™, Novell Netware™, Sun Microsystems Solaris™, OS/2™, BeOS™, Mach, Apache, OpenStep™, or another operating system or platform. In one implementation, the server may host an application comprising an Internet web system, an intranet system, or another system or application that can provide a hosted service. Additionally, the remote supervisory center 110 may comprise, include, or interface with one or more databases or other data storage platforms, which may use any suitable query formats or resources for storing and retrieving various types of emotional response test data, as described in greater detail herein.

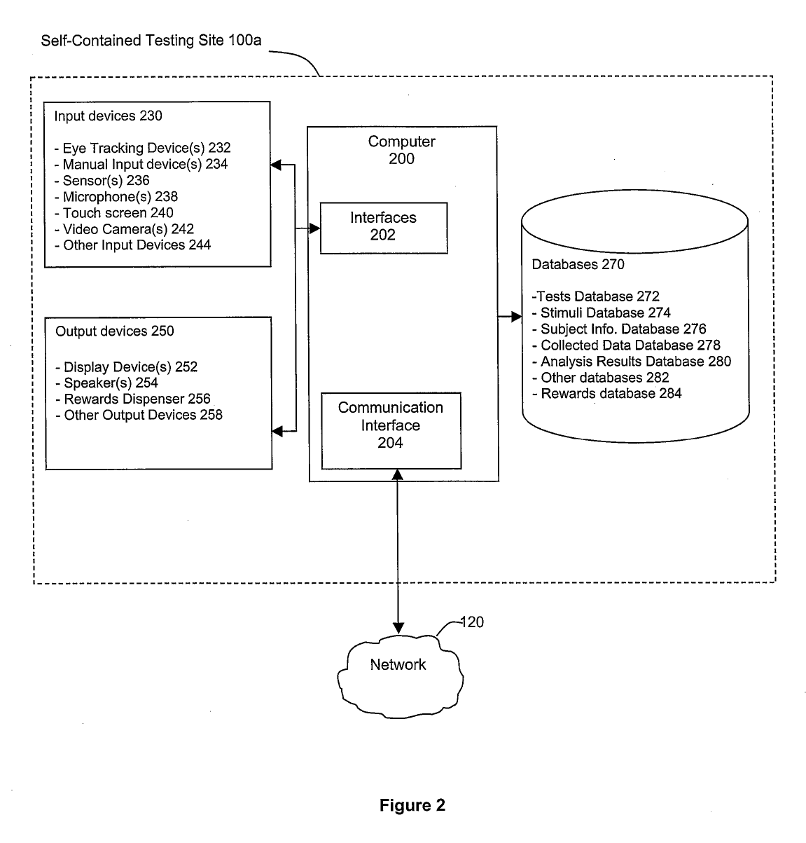

FIG. 2 illustrates an exemplary self-contained data collection system 100a, according to one aspect of the invention. As illustrated in FIG. 2, the self-contained data collection system 100a may comprise, among other things, at least one computer 200 coupled to one or more input devices 230 and one or more output devices 250 via one or more interfaces 202. The self-contained data collection system 100a may also interface with one or more databases 270, and may be communicatively coupled to a network 120 (e.g., to communicate with the remote supervisory center 110 described in connection with FIG. 1). Computer 200 may comprise any suitable combination of hardware, software, and/or firmware that can enable the features and functions described herein.

The one or more input devices 230 may comprise one or more of an eye tracking device 232, a manual input device 234, a sensor 236, a microphone 238, a touch-screen 240, a video camera 242, and/or any other input device 244 that can receive input from one or more subjects. The manual input device 234 may include one or more of a keyboard, a mouse, or another input device that enables subjects to manually input information to the computer 200.

Eye-tracking device 232 may comprise a camera or another known or future-developed eye-tracking device that records and tracks various eye properties of subjects (e.g. while the subject is being presented with one or more test stimuli). Examples of eye properties that may be tracked can include, blink rate, eye movement, pupil dilation, or gaze sequence, among others. In various implementations, the eye-tracking device 232 may be attached to a display device 252, integrated with the display device 252, or configured as a stand-alone device. The eye-tracking device 232 may interface with computer 200 via any suitable connection or interface. Various eye tracking devices, per se, are known.

The sensor 236 may include any one or more an emotion detection sensor, a biometric sensor, a physical attribute sensor, an environment sensor, a distance detection sensor, or another sensor or sensory device.

Emotion detection sensors may comprise, for example, physiological sensors such as galvanic skin response sensors, facial recognition sensors, heart rate sensors, sweat detection sensors, stress sensors, or any other sensors or future-developed sensors that can detect physiological responses from one or more subjects.

Biometric sensors may comprise, for example, one or more iris-scanning sensors, fingerprint-scanning sensors, thermal imaging sensors, or any other sensors or future-developed sensors that can acquire biometric information from the subjects.

Physical attribute sensors may comprise, for example, one or more weight sensors, height sensors, or any other suitable sensor or future-developed sensor that can measure physical attributes or other body metrics of the subjects.

Environment sensors may comprise, for example, one or more light-intensity sensors, background noise sensors, temperature sensors, smell sensors, or any other sensors or future-developed sensors that can measure various environmental parameters of self-contained testing system 100a.

Distance detection sensors may comprise, for example, one or more sensors that can measure a distance from the display device 252 and/or eye-tracking device 232 to a subject. In one implementation, the eye-tracking device 232 may itself operate as the distance detection sensor to measure the distance from the eye-tracking device 232 to the subjects. In one implementation, one or more display devices 252 may be placed at different distances from the subject to accommodate various types of testing. For example, a display device 252 may be placed relatively closer to the subject when displaying a single product (e.g., to test the subject's emotional response to the specific product). In another example, the display device 252 may be larger or placed farther away from the subject to test the subject's response to the product in a commercial context (e.g., the display device 252 may present a replica of a shelf of products that includes the product being tested). Additionally, in yet another example, one or more displays 252 may comprise a pull-down screen onto which one or more images or other stimuli may be projected. Other variations and examples will be apparent.

Microphone 238 may comprise, for example, any suitable device that enables the subjects to provide voice-activated input for responding to various instructions and messages, stimuli, and/or other information.

Touch-screen 240 may comprise any suitable device to accept manual input from the subjects via, for example, physical contact/pressure applied to the screen via the subjects' finger, a stylus, or another body part or apparatus. In one implementation, display device 252 may comprise, for example, a touch-screen monitor that can accept manual input from the subjects and present instructions, messages, stimuli, and/or other information to the subjects.

Video camera 242 may monitor the self-contained data collection system 100a either continuously or at certain times or intervals. The video camera 242 may capture images and/or videos, which may be stored locally at the self-contained data collection systems 100a and/or at remote supervisory center 110 for subsequent analysis, as needed. For example, when test results indicate that data may be suspect or unreliable, the images and/or videos that video camera 242 captured may be synchronized to the suspect or unreliable data. As such, the suspect or unreliable data may be reviewed at the self-contained data collection systems 100a and/or the remote supervisory center 110 to identify possible causes of the suspect data (e.g., the subjects failed to follow some or all of the required test protocols, or the test environment unduly influenced the subjects, etc.). In one implementation, when the test results indicate suspect or unreliable data, the thermal imaging sensor may capture a heat signature of the subjects in addition to the images and/or videos that the video camera 242 captured.

The various other input devices 244 may include, for example, card readers scanners, or other devices that can be used, for instance, to read subjects' drivers licenses, credit cards, and/or other cards, or to retrieve names, demographics, and/or other information regarding the subjects.

According to one implementation, the output devices 250 may include one or more of a display device 252, a speaker 254, a rewards dispenser 256, or other output devices 258.

Display device 252 may comprise one or more monitors, Cathode Ray Tube (CRT) displays, digital flat panel displays (e.g., LCD displays, plasma displays, etc.), or other display devices for presenting visual instructions, messages, stimuli, and/or other information to subjects. The display device 252 may comprise one or more external monitors, display screens, or other display devices for indicating whether the self-contained data collection system 100a is currently active, displaying welcome messages, or displaying other information (e.g., promising a reward for participating in the test). As such, emotional response testing may be administered at the self-contained data collection 100a in a manner designed to attract test subjects. In one implementation, the display device 252 may display messages for recruiting subjects of a specific targeted demographic (e.g., by displaying messages requesting subjects of a particular age group, gender, or other demographic to participate in the tests). In one implementation, one or more operators may be employed at the self-contained data collection system 100a to actively recruit subjects for one or more tests and to assist the subjects during administration of the tests.

Speaker 254 may comprise one or more speakers for audibly reproducing audio instructions, messages, stimuli, or other information to subjects.

Rewards dispenser 256 may dispense one or more incentives to the subjects, such as coupons, gift certificates, gift cards, or other incentives to subjects participating in a test. In one implementation, the incentives may be dispensed to the subjects when a test administered at the self-contained data collection system 100a terminates.

Databases 270 may comprise a tests database 272, a stimuli database 274, a subject information database 276, a collected data database 278, a results database 280, a rewards database 284, and/or other databases 282.

Tests database 272 may store one or more tests comprising any individual, series, or combination of stimuli that may be presented to a subject during an emotional response test. The tests database 272 may store information relating to target demographics defined for the tests, appropriate emotional profiles for the tests, and/or appropriate environmental parameters for the tests, among other things. For example, a given test may require self-contained data collection system 100a to be quiet and dimly lit while a subject is taking the test. In another example, a given test may require subjects to qualify prior to the test being administered, wherein an emotional segmentation process may determine whether the subjects have a suitable emotional profile or otherwise have emotional characteristics suitable for the test.

In one implementation, one or more test stimuli associated with one or more tests may be stored in stimuli database 274. In one implementation, additional stimuli that may not necessarily be associated with an emotional response test may also be stored in the stimuli database 274. As previously noted, the test stimuli presented to subjects may comprise any fixed or dynamic stimulus or stimuli relating to one or more of the subject's five senses (i.e., sight, sound, smell, taste, touch). The stimulus may comprise any real stimulus, or any analog or electronic stimulus that can be presented to the subject via known or future-developed technology. For example, visual stimuli may include, but are not limited to, pictures, artwork, charts, graphs, text, movies, multimedia or interactive content, or other visual stimuli. The stimuli may be recorded on any suitable media and may include live scenarios (real-time generation). Other test stimuli, such as aromas, may also be used either alone or in combination with other test stimuli. For example, an aroma synthesizer may be used to generate the aromas and the subjects' response to the aromas may then be evaluated.

According to one aspect of the invention, remote supervisory center 110 may comprise a master stimuli database (not illustrated) that stores one or more stimuli that may be presented to subjects participating in the tests being administered at any of the one or more self-contained data collection systems 100a-n.

In one implementation, information regarding test subjects may be stored in subject information database 276. Subject information may include, but is not limited to, demographic information (e.g., age, gender, race, etc.), identification information (e.g., name, iris scan, finger print, etc.), tests a subject is participating or has participated in, physical attribute information (e.g., height, weight, etc.), or other information. This information may be acquired, for example, via input received from the subjects using one or more of the aforementioned input devices 230, including various sensors (e.g., biometric sensors, physical attribute sensors, information readers, and/or other devices or sensors). Subject information profiles, including the acquired subject information, may be created for each subject participating in an emotional response test administered at the self-contained data collection system 100a, and these subject information profiles may also be stored in subject information database 276.

According to one aspect of the invention, initial emotional response data for the subjects may be acquired from the subjects via the aforementioned input devices 230 prior to administration of one or more emotional response tests. The initial emotional response data may be collected in response to one or more stimuli that may or may not be associated with the tests to be subsequently administered. The initial emotional response data may comprise, for example, data relating to properties of the subjects' eyes (e.g., pupil dilation, blink rate, eye movement, gaze sequence, etc.). The eye-tracking device 232 may acquire the data relating to the properties of the subjects' eyes, and the data may be analyzed in view of physiological conditions of the subjects acquired from one or more of the sensors and/or other information. The initial emotional response data may then be analyzed to determine the subjects' emotional characteristics (e.g., phobias, and/or other characteristics).

Collecting the initial emotional response data may include asking the subjects a series of questions and requesting the subjects to provide an input in response. From the subjects' responses to the questions, and from sensory information and eye-related information gathered from the subjects, various emotional response characteristics of the subjects may be determined (e.g., phobias, personality types, and/or other emotional characteristics). As such, emotional response profiles including the initial emotional response data and the emotional response characteristics may be created for each subject participating in the tests, and these emotional response profiles and emotional response characteristics may also be stored in the subject information database 276. The subject information acquired at each of the self-contained data collection systems 100a-n may be transmitted to remote supervisory center 110 in real-time, at any predetermined interval (e.g., hourly, daily, weekly, etc.), once a predetermined number of tests have been completed, or in other ways. The remote supervisory center 110 may include a master collected data database (not illustrated) for storing the data received from any of the one or more self-contained data collection systems 100a-n.

When administering tests at the self-contained data collection system 100a, one or more stimuli associated with the tests may be presented to the subjects, and data regarding the subjects' emotional responses to the presented stimuli may be collected. The collected data may comprise, for example, eye property data acquired via eye-tracking device 232 (e.g., pupil dilation, blink rate, eye movement, gaze sequence, or other eye properties), data regarding physiological conditions of subjects acquired from various sensor, data regarding the distance between the display device 252 and/or eye-tracking device 232 and each subject, among other things. In one implementation, this data may be stored locally in collected data database 278, and/or may be transmitted to remote supervisory center 110 for storage and/or subsequent analysis. The data collected at each of the self-contained data collection systems 100a-n may be transmitted to remote supervisory center 110 in real-time, at any predetermined interval (e.g., hourly, daily, weekly, etc.), once a predetermined number of tests have been completed, or in other ways. The remote supervisory center 110 may include a master collected data database (not illustrated) for storing the collected data received from any of the one or more self-contained data collection systems 100a-n.

The data collected at the self-contained data collection system 100a may be analyzed at the self-contained data collection system 100a (or elsewhere) and the results may be stored locally in an analysis results database 280 (or elsewhere). The results may then, in certain implementations, be transmitted to remote supervisory center 110 in real-time, at any predetermined interval (e.g., hourly, daily, weekly, etc.), once a predetermined number of tests have been completed, or in other ways. The remote supervisory center 110 may include a master collected data database (not illustrated) for storing the collected data received from any of the one or more self-contained data collection systems 100a-n. The remote supervisory center 110 may perform the analysis on the collected data received from the self-contained data collection system 100a, and the remote supervisory center 110 may further comprise a master analysis results database (not illustrated) for storing the results of analysis of collected data received from any of the one or more self-contained data collection systems 100a-n.

According to one aspect of the invention, rewards database 284 may store various incentives that may be provided to subjects as incentives to participate in a test and/or as a reward for participation in a test. Examples of incentives may include, but are not limited to, one or more coupons, gift certificates, gift cards, or other incentives. Additional information may be stored in rewards database 284 including, but not limited to, which incentives are associated with which tests, which incentives should be made available for which test subjects, which subjects have received which incentives, and other information. The remote supervisory center 110 may comprise a master rewards database (not illustrated) that stores incentives along with any or all of the information described above with regard to rewards database 284.

In various implementations, any number of entities may provide rewards to the master rewards database at remote supervisory center 110 and/or or to rewards database 284 at self-contained data collection system 100a. For example these entities may include, but are not limited to, coupon issuers or distributors, or an entity requesting a test (e.g., a makeup company wishing to test an emotional response to a new advertisement for a particular product may provide coupons for the product), among others.

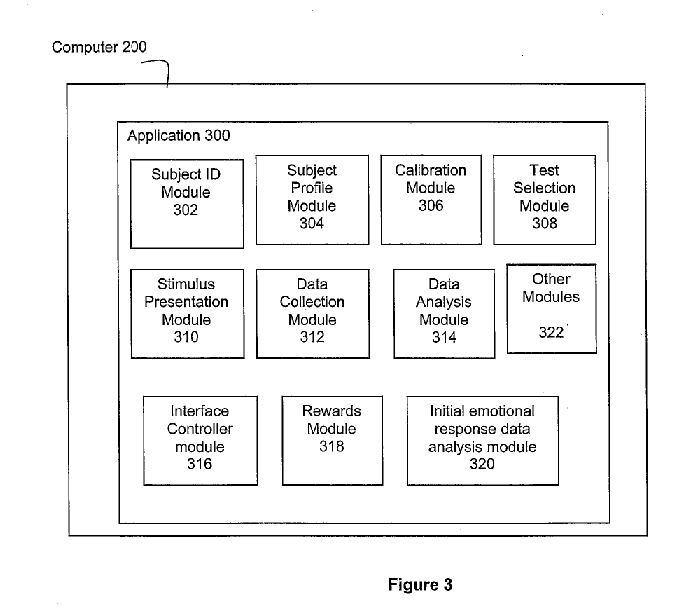

According to one aspect of the invention, as illustrated in FIG. 3, an application 300 may execute on the computer 200 associated with self-contained data collection system 100a. The application 300 may comprise one or more software modules that enable the various features and functions of the invention, including one or more of calibration, identity verification, profile creation/retrieval, test selection, stimuli presentation, data collection, data analysis, initial emotional response data analysis, or other functions.

Non-limiting examples of the modules in application 300 may include one or more of a subject ID module 302, a subject profile module 304, a calibration module 306, a test selection module 308, a stimuli presentation module 310, a data collection module 312, a data analysis module 314, an interface controller module 316, a rewards module 318, initial emotional response data analysis module 320, or other modules 322. In various implementations, one or more of the modules comprising application 300 may be combined, and for various purposes, all modules may or may not be necessary. It will further be recognized that, in various implementations, any of the features and functions that the modules of application 300 enable may also be provided through similar modules or a similar application at remote supervisory center 110. In one implementation, for example, the modules illustrated in FIG. 3 and described herein may be run solely on the computer 200 at the self-contained data collection system 100a, solely at remote supervisory center 110, or various combinations thereof.

In one implementation, the subject ID module 302 may verify an identity of one or more subjects participating in tests administered at the self-contained data collection system 100a. In one exemplary implementation, subject ID module 302 may utilize biometric information (e.g., iris scan images, fingerprint images) acquired from the subjects via biometric sensors to verify the identity of the subjects.

In one implementation, the subject profile module 304 may enable new subject information profiles and/or emotional response profiles to be created, and may further enable existing subject information profiles and/or emotional response profiles to be retrieved and/or modified. For example, subject profile module 304 may prompt subjects to input personal information including, but not limited to, name, age, gender, various physical attributes (e.g., height, weight, etc.), or other information. Subject profile module 304 may also acquire information regarding the physical attributes of subjects from physical attribute sensors. Subject profile module 304 may acquire biometric information (e.g., iris scan images, fingerprint images, etc.) for subjects via one or more biometric sensors. Subject profile module 304 may additionally process subject information (e.g., name, demographic information, and/or other information) acquired from information cards (e.g., drivers licenses, credit cards, or other cards) via one or more information readers.

In one implementation, based on at least a portion of the information acquired from the various sources mentioned above, subject profile module 304 may determine whether the subject is a new subject for whom a subject information profile and/or an emotional response file must be created, or a returning subject for whom the subject information profile and/or the emotional response profile already exists in subject information database 276.

When the subject is a new subject, subject profile module 304 may register the subject and create the subject information profile for the subject using at least a portion of the information acquired from various sources mentioned above, and may create the emotional response profile using at least a portion of information acquired via manual input, eye-tracking device 232, emotion detection sensors, and/or other input devices or sensors. Initial emotional response data analysis module 320 may then analyze the subjects' collected initial emotional response data and the subject's emotional characteristics (e.g., phobias, personality type, and/or other characteristics) may be determined based on the analysis. Subject profile module 304 may therefore create an emotional response profile for the subject based on the initial emotional response data acquired from various sources mentioned above and the determined emotional characteristics.

When the subject is a returning or existing subject, subject profile module 304 may retrieve an existing subject information profile and/or an existing emotional response profile for the subject and enable the subject to modify the retrieved subject information profile and/or the retrieved emotional response profile to add, modify, delete, or otherwise update the subject information profile and/or the emotional response profile, as necessary. In one implementation, subject profile module 304 may also collect initial emotional response data to have initial emotional response data analysis module 322 analyze the collected emotional response data for a returning subject. The subject profile module 304 may then update the subject's existing emotional response profile, as necessary.

According to one aspect of the invention, calibration module 306 may employ various calibration processes. For example, the calibration module 306 may adjust various sensors to an environment of a self-contained data collection system 100a, adjust various sensors or devices to a subject within the self-contained data collection system 100a, and determining a baseline emotional level for the subject within the self-contained data collection system 100a, among other calibrations. Adjusting or otherwise calibrating to the particular environment at the self-contained data collection system 100a may include measuring ambient conditions or parameters (e.g., light intensity, background noise, temperature, smell, etc.), and if necessary, adjusting the ambient conditions or parameters to ensure that meaningful data can be acquired.

According to one aspect of the invention, one or more devices or sensors may be adjusted or calibrated to the subject. For the acquisition of eye property data, for example, the subject may be positioned (e.g., sitting, standing, or otherwise) so that eye-tracking device 232 has an unobstructed view of either the subject's left eye, right eye, or both eyes. Calibration module 306 may generate calibration-related instructions or messages that may be presented to the subject via one or more of the output devices (e.g., the subject may be instructed to move closer to or further from the eye-tracking device 232). Eye-tracking device 232 may also self-adjust to ensure an unobstructed view of either the subject's left eye, right eye, or both eyes. Eye-tracking device 232 may be calibrated to ensure that the image of a single eye or both eyes of a subject are clear, focused, and suitable for tracking eye properties of interest.

Calibration module 306 may also enable calibration of any number of other sensors or devices (e.g., other emotion detection sensors, distance detection sensors, biometric sensors, microphones, or other sensors/devices). As such, calibration module 306 may ensure that accurate data can be acquired when administering tests at the self-contained data collection system 100a. For example, one or more microphones 238 for speech or other audible input may be calibrated to ensure that a subject's speech is acquired under optimal conditions, at an adequate level, or otherwise. During the calibration, distance detection sensors may determine the distance between display device 252 and/or eye-tracking device 232 and a subject, and may further establish the determined distance as a reference distance.

In one implementation, calibration module 306 may also attempt to adjust a subject's emotional level to ensure that the subject is in an emotionally neutral state prior to presenting stimuli associated with a test to be administered (e.g., a calm and soothing voice may instruct the subject to close their eyes and relax for a few moments). Calibration data associated with the subject may also be stored in the subject's subject information profile in subject information database 276, or in another database.

Additional details on these and other functions performed during calibration are discussed in U.S. patent application Ser. No. 11/522,476, entitled “System and Method for Determining Human Emotion by Analyzing Eye Properties,” filed Sep. 18, 2006 and published as U.S. Patent Application Publication No. 2007/0066916 on Mar. 22, 2007, and in U.S. patent application Ser. No. _______, entitled “System and Method for Calibrating and Normalizing Eye Data in Emotional Testing,” filed on even date herewith (Attorney Docket No. 067578-0360357), the disclosures of which are hereby incorporated by reference in their entireties.

According to aspect of the invention, test selection module 308 may automatically select one or more tests from tests database 272 based on subject information that a subject enters, and/or that is acquired about a subject. Based at least on this information, test selection module 308 may determine one or more tests in tests database 272 that may be appropriate for the subject. For example, one or more tests may be selected based on the subject's demographic or other criteria. For example, a makeup company may wish to test the emotional responses of a targeted demographic to a new advertisement for a particular product (e.g., girls ages sixteen to twenty-five). The self-contained testing system 100a may therefore be located in a shopping mall, for example, to entice potential subjects as volunteers for a test in exchange for some reward (e.g., a free sample of the product). If a volunteer subject is determined to be a twenty-year old female based on information the subject enters and/or that is acquired about the subject from subject profile module 304, then tests database 272 may select the test corresponding to the makeup company to be administered to the twenty-year-old female subject. Test selection module 308 may similarly select one or more tests from tests database 272 based on a subject's emotional characteristics, as maintained in emotional response profiles. For example, from the emotional profiles associated with a given test, it may be determined that the test should be administered only to subjects having particular emotional characteristics.

According to one aspect of the invention, the test selection process may be partially automated. For example, test selection module 308 may determine a list of tests to be presented based on at least a portion of a subject's information and/or subject's emotional characteristics. The list of tests may be presented to the subject via display device 252, speaker 254, or another device, and the subject may then select one or more of the tests from the list for which the subject would desire to participate. In one implementation, test selection module 308 may determine whether to adjust the selected tests administered to the subject, and/or whether additional tests should be administered to the subject after completion of a particular test.

In one implementation, stimuli presentation module 310 may facilitate presentation of one or more stimuli associated with the tests that test selection module 308 selects. The stimuli may be retrieved from the stimuli database, and may be presented to the subject via one or more of display device 252, speaker 254, or any other devices. The stimuli presentation module 310 may also facilitate presentation of stimuli that may not necessarily be associated with the tests. For example, these stimuli may be presented to the subjects to collect initial emotional response data from the subject prior administering tests that the test selection module 308 selects.

According to one aspect of the invention, data collection module 312 may collect data regarding the emotional responses of subjects to the presented stimuli that are associated with the tests that test selection module 308 selects. Data collection module 312 may direct the collected data for storage either locally in collected data database 270, or remotely in the master collected data database at remote supervisory center 110, or both.

According to one aspect of the invention, data analysis module 314 may analyze the collected emotional response data that the data collection module 312 collects to determine the emotional impact, if any, that the presented stimuli had on test subjects. For example, data analysis module 314 may analyze eye property data to determine one or more emotional components measured from the subject (e.g., emotional valence, arousal, category, type, etc.). Aspects of this analysis are described in greater detail in U.S. Patent Application Publication No. 2007/0066916, which has been previously incorporated by reference.

In one implementation, distance data that a distance detection device collects may be analyzed to determine any changes in the distance between the subject and the display device 252 and/or eye-tracking device 232 during a test. For example, a shorter distance may represent a subject's movement towards the device, possibly indicating an increased interest in and/or a positive response to the presented stimuli. In contrast, a larger distance may represent a subject's movement away from the device and may indicate a disinterest in and/or negative response to the presented stimuli. The physiological data from the emotion detection sensors may also be analyzed to determine any changes in the physiological conditions of the subjects from prior to, during, or after testing, or in other ways. Data analysis module 314 may direct the results of the analysis for storage locally in analysis results database 278, or remotely in the master analysis results database at remote supervisory center 110, or both.

According to one aspect of the invention, based on the results of the analysis, test selection module 308 may determine whether to adjust subsequent tests to be administered, and/or whether additional tests should be administered to the subjects. For example, a subject's interest level, as determined above, may be used as a factor in determining whether to administer additional tests or adjust subsequent tests.

According to one aspect of the invention, video and/or image data obtained from video camera 242 may be synchronized to and associated with acquired subject data and/or test data. In this regard, more or more quality controls may be implemented. In particular, video and/or image data may be analyzed for each subject, for a predetermined number of subjects, or for a random selection of subjects to determine whether subjects have performed any anomalous activities that raise quality control concerns. For example, when the video and/or image data from the video camera 242 clearly depicts that the subject is actually of a younger or older age, the age information that the subject input may be determined as erroneous. In this case, the emotional response data collected during test administration and the subsequent analysis of the collected emotional response data may also be determined as erroneous and flagged as such.

In addition to enabling quality control measures, video and/or image data obtained from video camera 242 may be utilized to explore potential causes of suspect or unreliable data. For example, if test results indicate suspect or unreliable data, images and or videos captured by video camera 242 and synchronized to the collected data may be reviewed to identify a cause of the suspect or unreliable data (e.g., the subject failed to follow some or all of the required test protocols). In one implementation, when test results indicate suspect or unreliable data, a heat signature of the subject may be captured using the thermal imaging sensor in addition to the images and/or videos captured by video camera 242.

According to one aspect of the invention, rewards module 318 may determine which incentives should be provided to which subjects as incentives to participate in a test and/or as a reward for participation. In one implementation, initial emotional response data analysis module 322 may analyze a subject's collected initial emotional response data and the subject's emotional characteristics (e.g., phobias, personality type, and/or other characteristics) may be determined based on the analysis.

In one implementation, biometric information for subjects acquired at a self-contained data collection system 100a may be transmitted to remote supervisory center 110, and the identity of the subjects may be verified at remote supervisory center 110. Various other acquired subject information relating to subjects may also be transmitted to the remote supervisory center 110, wherein at least a portion of the acquired information may be used to register a new subject, create a subject information profile for the new subject, retrieve an existing subject information profile for an existing subject, or perform other functions. In one implementation, subjects' collected initial emotional response data and/or emotional characteristics may be also transmitted to the remote supervisory center 110, wherein at least a portion of the initial emotional response data and/or emotional characteristics may be used to create emotional response profiles for the subjects, or perform other functions. In one implementation, calibration of the sensors, devices, subjects, environment, and other test characteristics at the self-contained data collection system 100a may be performed remotely (e.g., under the direction of the remote supervisory center 110).

According to one aspect of the invention, test selection may be performed remotely at remote supervisory center 110. The remote supervisory center 110 may utilize at least a portion of acquired subject information received from self-contained data collection system 100a to determine one or more tests that may be appropriate for the subjects, and to select these tests from the master tests database. According to one aspect of the invention, remote supervisory center 110 may utilize at least a portion of subjects' collected initial emotional response data and/or emotional characteristics to determine one or more tests that may be appropriate for the subjects, and to select these tests from the master tests database. Remote supervisory center 110 may then transmit the selected tests to the self-contained data collection system 100a to be administered to the subjects at the self-contained data collection system 100a.

In one implementation, a test operator located at remote supervisory center 110 may supervise all or a portion of a test administered for a subject at a self-contained data collection system 100a. The operator may also provide instructions and/or other information to the subject via any number of the system components (e.g., display device, speakers, etc.), as described in greater detail above. The test operator may supervise a test via video camera 242, and may have real-time access to any and all data from any phase of the testing (e.g., acquisition of a subject's physical attribute data, control of environmental parameters at the testing system, calibration, etc.). Depending on the volume of tests to be administered and/or the number of self-contained data collection systems 100a-n, a plurality of remote supervisory centers 110 may exist, and each may or may not be staffed with any number of test operators (e.g., similar to operations at a call center). Various alternative implementations may also be utilized.

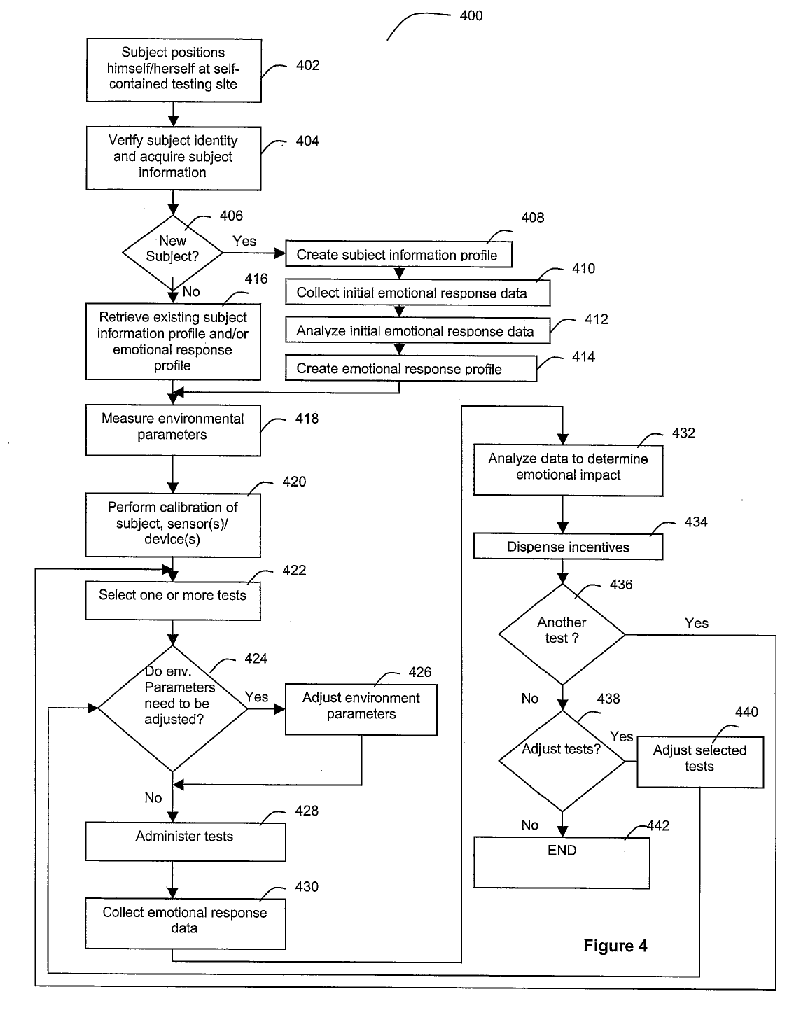

FIG. 4 illustrates an exemplary process for operating a self-contained data collection system. The operations described herein may be accomplished using some or all of the features and components described in greater detail above and, in some implementations, various operations may be performed in different sequences. In some implementations, additional operations may be performed along with some or all of the operations shown in FIG. 4, or one or more operations may be performed simultaneously. Accordingly, the operations described herein are to be regarded as exemplary in nature.

In an operation 402, upon arriving at or otherwise accessing a self-contained testing system, a subject may position himself or herself (e.g., sitting, standing, or otherwise) in front of a display device and/or an eye-tracking device.

In an operation 404, information about the subject (e.g., name, age, gender, physical attributes, biometric information, or other information) may be acquired. In one implementation, the subject may be prompted to enter the information manually. Information about the subject's physical attributes (e.g., height, weight, etc.) may also be acquired via one or more physical attribute sensors. Biometric information (e.g., iris scan images, fingerprint images, etc.) for the subject may also be acquired from one or more biometric sensors. Information may also be acquired from the subject from various information cards (e.g., drivers licenses, credit cards, etc.) via one or more information readers. The acquired biometric information may be used, for example, to verify the identity of a returning subject, or to create a profile of a new subject.

In an operation 406, at least a portion of the information acquired in operation 402 may be used to determine whether a subject is a new subject, or a returning or existing subject.

If a determination is made in operation 406 that the subject is a new subject, the new subject may be registered and a subject information profile may be created for the new subject in an operation 408.

In an operation 410, initial emotional response data for the subject may be collected from the subject using one or more input devices, eye-tracking devices, emotion detection sensors, and/or other sensors. The initial emotional response data may be collected in response to one or more stimuli that may not be associated with tests. The initial emotional response data may comprise, for example, eye property data (e.g., pupil dilation, blink rate, eye movement, or other eye properties) acquired via the eye-tracking device, data regarding physiological conditions of subjects acquired from the emotion detection sensors, and/or other data. Asking the subject a series of questions and requesting input from the subject may also be performed when collecting the initial emotional response data.

In an operation 412, the initial emotional response data may be analyzed to determine a subject's emotional characteristics. The emotional characteristics, may include, but are not limited to, phobias, personality type, and/or other characteristics.

In an operation 414, an emotional response profile may be created for the new subject using at least a portion of the initial emotional response data and/or the subject's emotional characteristics.

If a determination is made in operation 406 that the subject is a returning or existing subject, an existing subject information profile and/or emotional response profile for the subject may be retrieved from the subject information database in an operation 416.

In an operation 418, various environmental parameters (e.g., light intensity, noise, temperature, smell, or other parameters) of the self-contained testing system may be measured.

In an operation 420, if necessary, various calibration processes may be implemented to ensure suitability of testing conditions. Calibration may comprise, for example, adjusting various sensors or devices to the subject at the self-contained data collection system, as well as determining a baseline emotional level for the subject.

In an operation 422, one or more tests may be selected for the subject based on at least a portion of information acquired or retrieved for the subject. In one implementation, one or more tests may be selected for the subject based on the subject's emotional characteristics. In one implementation, test selection operation 422 may be performed automatically. In one implementation, test selection operation 422 may be partially automatic, wherein a list of tests may be presented to the subject from which the subject selects one or more tests in which to participate.

In an operation 424, a determination may be made as to whether environmental parameters (e.g., light intensity, noise, temperature, etc.) for the self-contained data collection system need to be adjusted based on the one or more selected tests. If a determination is made in operation 424 that one or more environmental parameters need to be adjusted, such adjustment may occur in an operation 426.

If a determination is made in operation 424 that no environmental parameters need to be adjusted to match the environmental parameters associated with a selected test, then processing may continue to an operation 428, where a selected test may be administered. For example, in operation 428, the subject may be presented with one or more stimuli associated with the selected test.

In an operation 430, emotional response data for the subject is collected during the test. As previously described, collected emotional response data may comprise eye property data, data concerning one or more physiological attributes of the subject from one or more emotion detection sensors, data regarding the distance between the display device 252 and/or eye tracking devices 232 and the subject, and/or other emotional response data.

In an operation 432, the collected data is analyzed to determine the emotional impact that the one or more presented stimuli had on the subject.

Upon completion of a test, one or more incentives may be dispensed for the subject in an operation 434.

In an operation 436, a determination may be made as to whether an additional test is to be administered. In one implementation, the determination as to whether an additional test is to be administered is made based on the results of the analysis performed in operation 432. If a determination is made in operation 436 that an additional test is to be administered, processing may return to operation 422, otherwise if a determination is made in operation 436 that no additional tests are to be administered, processing may continue to an operation 438.

In operation 438, a determination may be made as to whether the one or more tests selected in operation 422 should be adjusted. In one implementation, the determination is made based on the results of the analysis performed in operation 432. If a determination is made in operation 438 that one or more selected tests should be adjusted, such adjustment is performed in an operation 440, and processing may then return to operation 424. Otherwise, if a determination is made in operation 438 that no adjustment should be made, processing may end at operation 442.

Aspects and implementations may be described as including a particular feature, structure, or characteristic, but every aspect or implementation may not necessarily include the particular feature, structure, or characteristic. Further, when a particular feature, structure, or characteristic has been described in connection with an aspect or implementation, it will be understood that such feature, structure, or characteristic may be included in connection with other aspects or implementations, whether or not explicitly described. Thus, various changes and modifications may be made to the preceding description without departing from the scope or spirit of the invention, and the specification and drawings should therefore be regarded as exemplary only, and the scope of the invention determined solely by the appended claims.

What is claimed is:

1. A self-contained computer-implemented data collection system for emotional response testing, comprising:
a calibration module configured to adjust one or more devices used in an emotional response test, wherein the devices are adjusted according to an environment and a subject of the emotional response test;
a subject module configured to identify the subject and determine one or more profiles for the identified subject;
a test selection module configured to select one or more tests from an emotional response test database based on the profiles for the identified subject;
one or more output devices configured to present one or more stimuli to the subject during the selected emotional response test; and
one or more input devices configured to receive input from the subject during the emotional response test, wherein at least one of the input devices are configured to measure and process eye data to determine the subject's emotional response to the stimuli presented during the emotional response test.

2. The system of claim 1, wherein the profiles for the identified subject include at least one of an information profile or an emotional response profile.

3. The system of claim 1, wherein the input devices include at least one of an emotion detection sensor, a biometric sensor, a physical attribute sensor, an environment sensor, a distance detection sensor, or another sensor or sensory device.

4. The system of claim 1, wherein the measured and processed eye data includes one or more of pupil dilation, blink rate, eye movement, gaze sequence, or other eye properties.

5. The system of claim 1, further comprising a rewards module configured to dispense one or more awards to the subject upon completion of the emotional response test.

6. The system of claim 1, further comprising a collected data database configured to store data regarding the subject's emotional responses to the stimuli presented during the emotional response test.

7. The system of claim 6, further comprising an analysis results database configured to analyze the data in the collected data database.

8. The system of claim 1, further comprising an interface to a remote supervisory center, wherein the self-contained data collection system and the remote supervisory center periodically exchange information relating to emotional response testing.

9. A self-contained computer-implemented data collection method for emotional response testing, comprising:
adjusting one or more devices used in an emotional response test, wherein the devices are adjusted according to an environment and a subject of the emotional response test;
identifying the subject to determine one or more profiles for the identified subject;
selecting one or more tests from an emotional response test database based on the profiles for the identified subject;
presenting one or more stimuli to the subject during the selected emotional response test; and
receiving input from the subject during the emotional response test, wherein at least one of the input devices are configured to measure and process eye data to determine the subject's emotional response to the stimuli presented during the emotional response test.

10. The method of claim 9, wherein the profiles for the identified subject include at least one of an information profile or an emotional response profile.

11. The method of claim 9, wherein the input is received using at least one of an emotion detection sensor, a biometric sensor, a physical attribute sensor, an environment sensor, a distance detection sensor, or another sensor or sensory device.

12. The method of claim 9, wherein the measured and processed eye data includes one or more of pupil dilation, blink rate, eye movement, gaze sequence, or other eye properties.

13. The method of claim 9, further comprising dispensing one or more awards to the subject upon completion of the emotional response test.

14. The method of claim 9, further comprising storing data regarding the subject's emotional responses to the stimuli presented during the emotional response test.

15. The method of claim 14, further comprising analyzing the data in the collected data database.

16. The method of claim 9, further comprising periodically exchanging information relating to emotional response testing with a remote supervisory center.
